(19) United States
(12) Patent Application Publication
(10) Pub. No.: US 2012/0260922 A1
(43) Pub. Date: Oct. 18, 2012

(54) TOPICAL USE OF HYDROXYTYROSOL AND DERIVATIVES FOR THE PREVENTION OF HIV INFECTION

(76) Inventors: Eduardo Gómez-Acebo, Madrid (ES); José Alcami Pertejo, Madrid (ES); David Aunon Calles, Sangonera la Verde-Murcia (ES)

(21) Appl. No.: 13/513,061

(22) PCT Filed: Dec. 1, 2010
(86) PCT No.: PCT/EP10/68673

§ 371 Date: Jun. 25, 2012

(30) Foreign Application Priority Data
Dec. 1, 2009 (EP) 09382267.4

(51) Int. Cl. A61F 6/04; A61P 31/18; A61P 31/00; A61K 31/05

(52) U.S. Cl. 128/844; 514/731

(57) ABSTRACT

The present invention is directed to a topical pharmaceutical composition which comprises a compound of formula (I), such as hydroxytyrosol, or a pharmaceutically acceptable salt, solvate, prodrug or isomer thereof, and a pharmaceutically acceptable carrier

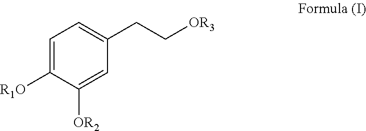

wherein R1, R2 and R3 take different values, and the use of said composition as 10 medicament in the prevention of sexually transmitted diseases (STDs), such as HIV-infection.

### FIELD OF THE INVENTION

This invention relates to topical microbicide pharmaceutical compositions that are useful for the prevention of sexually transmitted infections, particularly for HIV preventive therapy, and to their uses.

### BACKGROUND OF THE INVENTION

HIV is the acronym of the Human Immunodeficiency Virus, which is the aetiological infectious agent for the Acquired Immunodeficiency Syndrome (AIDS). HIV infects human and primate immune system cells, disturbing or abolishing their function and causing a progressive impairment of the immune system, which results in “immunodeficiency”.

Since 1981, when the first human case infected with HIV was reported, around 60 million people have been infected with HIV, and among them, 20 million people have died of HIV-related causes. In many developed countries, availability of combined antiretroviral treatments has led to spectacular decreases in mortality and morbidity regarding HIV/AIDS. As a consequence, there are more HIV-infected people who can enjoy better health conditions and an increased life expectancy. However, the dimensions of the epidemic remain staggering. In 2007 alone, 33 million people were living with HIV, 2.7 million people became infected with the virus, and 2 million people died of HIV-related causes. The situation in developing countries is in strong contrast with the described for the developed world. Mainly in Africa, where access to basic preventive care against the infection as well as to its treatment are limited and therefore AIDS progress and death casualties are dramatically higher.

Prevention is clearly the key to halting the progress of HIV. Since sexual transmission is by far the most common route of HIV infection, and therefore also opening the door for further infections, the promotion of safer sexual behaviours critical for preventing such transmission and other sexually transmitted diseases (STDs).

However, current prevention programmes have had disappointing impacts, in large part because of the politicization of prevention, and in particular the controversies surrounding the promotion of condoms, despite the proven efficacy of condoms as mitigation instrument against HIV infection, or the limited access to them particularly in Africa and other undeveloped regions. Hence, regarding existing policies and prevention programmes, the specific local cultural, political and material circumstances will influence the content of any particular programme. It is worth mentioning that there is a complex relationship between poverty and HIV transmission that has resulted in a vicious circle aggravated by poverty.

Biological and physiological factors are important for HIV spread. Among them, a critical one is the fact that women's physiology puts them at greater risk of becoming infected during unprotected vaginal intercourse than men. Girls and young women face an especially high risk of infection during unprotected sex with an HIV-positive man because the lining of the neck of the womb is not fully developed (UNAIDS 2008 Report on the global AIDS epidemic).

Once the individual has been infected by HIV, therapy with antiretroviral drugs (ARVs)—which significantly delay the progression of HIV to AIDS and allow people living with HIV to live relatively normal, healthy lives—is at present available since around 1996, mostly in developed countries. Distributing these drugs in sufficient amounts requires money, a well-structured health system and a sufficient supply of healthcare workers providing treatment and care to those living with HIV. This is not the case in the majority of developing countries.

Based on the above reported situation, there is an urgent need for preventing measures, that woman can easily handle and use without depending on the man's preventing methods and furthermore, could be accepted by the cultural environment.

Efforts are under way to develop a microbicide useful for women, for example a gel or cream that can be applied topically to the vagina in much the same way as today's spermicides (see for example Mc Gowan I. Curr. Opin. Infec. Dis. 2009: “Microbicides for HIV prevention: Reality or hope?”).

However, many promising candidates are discarded due to safety issues or lack of efficacy. There is yet no effective methods and compositions that solve the above mentioned need for a HIV prevention agent.

Hydroxytyrosol (HT; CAS Registry number [10597-60-1]), also known as 3-hydroxytyrosol, 3,4-dihydroxyphenyl ethanol (DOPET) or 4-(2-hydroxyethyl)-1,2-benzenediol, is a natural occurring phytochemical compound mainly found in olive oil that shows strong antioxidant properties by scavenging oxygen radicals in vitro and in vivo. Different biological activities have been described for this compound such as: inhibition of low density lipoprotein oxidation, inhibition of platelet aggregation, protection against DNA damage, antiinflammatory activity and microbicide.

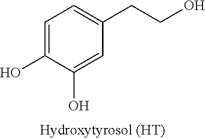

Recently, the dose-dependent inhibitory effect of HT on in vitro HIV-integrase activity has been described (Huang S L, Huang P L, Zhang D, Lee J W, Bao J, Sun Y, Chang Y T, Zhang J, Juang P L. Discovery of small-molecule HIV-1 fusion and integrase inhibitors oleuropein and hydroxytyrosol: Part II. Integrase inhibition. Biochem. Biophys. Res. Comm. 354: 879-884, 2007). The mechanism of action has been elucidated and reported. The o-dihydroxyphenol ring of HT binds to the integrase region II with strong H-bond interactions with F139 and nearby T115, and weak interactions with E318 and Q148. Since the dihydroxyphenol ring is capable of binding both regions I and II from integrase, HT is supposed to maintain the ability to bind to the integrase active site even if mutations occur. Thus, importantly, the likelihood of resistance development should be less than inhibitors that bind to a single site.

However, the inhibitory effect against HIV-integrase activity, which might be useful in the systemic treatment of infected individuals, does not suggest that the compound could also be useful in preventing HIV infections by topical application.

### SUMMARY OF THE INVENTION

The present inventors have surprisingly found that 3,4-dihydroxyphenyl ethanol and its derivatives are useful, when applied topically, as microbicide for preventing HIV-infection, as well as other sexually transmitted diseases (STDs) caused by fungi, bacteria or viruses. Importantly, hydroxytyrosol and its derivatives, when administered as microbicides for topical use, are a cheap and easy way to use as a prevention method against HIV-transmission and infection.

Thus, in one aspect, the present invention is directed to a topical pharmaceutical composition comprising a compound of formula (I):

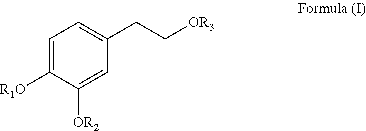

wherein

R₁, R₂ and R₃ are independently selected from the group consisting of hydrogen, substituted or unsubstituted alkyl, substituted or unsubstituted alkenyl, substituted or unsubstituted alkynyl, substituted or unsubstituted aryl, substituted or unsubstituted heterocyclyl, ORₐ, SRₐ, SORₐ, SO₂Rₐ, OSO₂Rₐ, OSO₃Rₐ, NO₂, NHRₐ, N(Rₐ)₂, ═N—Rₐ, N(Rₐ)CORₐ, N(CORₐ)₂, N(Rₐ)SO₂R′, N(Rₐ)C(═NRₐ)N(Rₐ)Rₐ, CN, halogen, CORₐ, COORₐ, OCORₐ, OCOORₐ, OCONHRₐ, OCON(Rₐ)₂, CONHRₐ, CON(Rₐ)₂, CON(Rₐ)ORₐ, CON(Rₐ)SO₂Rₐ, PO(ORₐ)₂, PO(ORₐ)Rₐ, PO(ORₐ)(N(Rₐ)Rₐ) and aminoacid ester;

each of the Rₐ groups is independently selected from the group consisting of hydrogen, substituted or unsubstituted alkyl, substituted or unsubstituted alkenyl, substituted or unsubstituted alkynyl, substituted or unsubstituted aryl, and substituted or unsubstituted heterocyclyl,

or a pharmaceutically acceptable salt, solvate, prodrug or isomer thereof, and a pharmaceutically acceptable carrier.

In another aspect, the present invention is also directed to a compound of general formula (I), or a pharmaceutically acceptable salt, solvate, prodrug, or isomer thereof for use as a medicament, particularly for the prevention of sexually transmitted diseases (STDs), preferably for the prevention of HIV-infection.

In another aspect, the present invention is also directed to the use of a compound of general formula (I) or a pharmaceutically acceptable salt, solvate, prodrug, or isomer thereof, in the preparation of a medicament for the prevention of sexually transmitted diseases (STDs), preferably for the prevention of HIV-infection.

Another aspect of the present invention is related to the method of preventing a sexually transmitted disease (STD), preferably HIV-infection, in a patient, notably a human, said method comprising administering to the patient a therapeutically or prophylactically effective amount of a compound of general formula (I) or a pharmaceutically acceptable salt, solvate, prodrug, or isomer thereof.

### BRIEF DESCRIPTION OF THE FIGS.

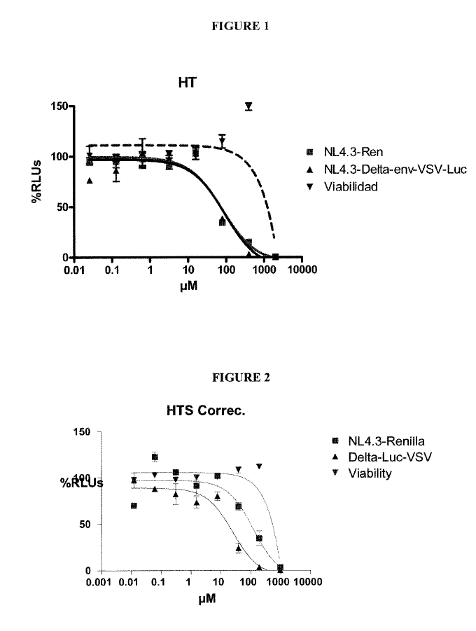

FIG. 1. Antiviral activity of hydroxytyrosol (HTS) in the infection of MT-2 cell line with X4-tropic virus infections (NL.4.3 Ren) and viruses pseudotyped with the VSV envelope (delta Luc). The viability was assessed on non-infected cells.

FIG. 2. Antiviral activity of hydroxytyrosol (HTS) after correcting the concentration in X4-tropic virus infections (NL.4.3 Ren) and viruses pseudotyped with the VSV envelope (delta Luc). The viability was assessed on non-infected cells.

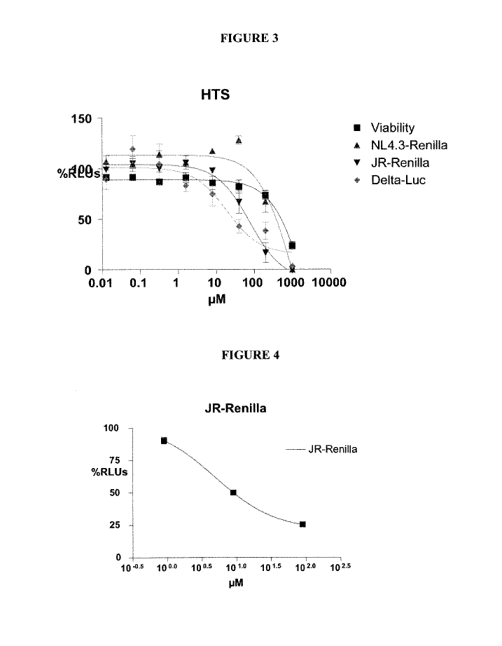

FIG. 3. Antiviral activity of hydroxytyrosol (HTS) in the infection of PBCM's with X4-tropic virus infections (NL.4.3 Ren), R5-tropic virus (JR-Ren) and viruses pseudotyped with the VSV envelope (delta Luc). The viability was assessed on non- infected cells.

FIG. 4. Antiviral activity of hydroxytyrosol (HTS) in the infection of PBCM's with R5-tropic viruses mediated with DC-SIGN+ cells (RAJI DC-SIGN).

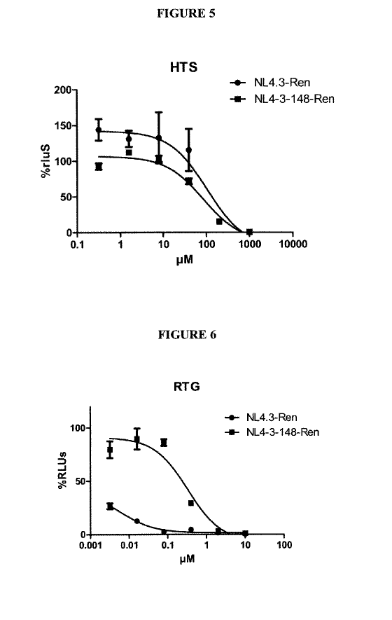

FIG. 5. Antiviral activity of hydroxytyrosol (HTS) against wild type and raltegravir resistant virus.

FIG. 6. Antiviral activity of raltegravir (control) against wild type and raltegravir resistant virus.

### DETAILED DESCRIPTION OF THE INVENTION

Topical pharmaceutical compositions useful for preventing HIV-infection as well as other sexually transmitted diseases (STDs) caused by fungi, bacteria or viruses, according to the present invention, comprise a compound of formula (I), or mixtures thereof, a pharmaceutically acceptable salt, solvate, prodrug, or isomer thereof together with a pharmaceutically acceptable carrier. They are useful as prophylactic agents, to prevent HIV infection in humans.

In order to facilitate the comprehension of the present invention, the meanings of some terms and expressions as used in the context of the invention are included herein:

“Microbicide” is any compound or substance whose purpose is to reduce the infectivity of microbes, such as viruses or bacteria.

A “prophylactic” is a medication or a treatment designed and used to prevent a disease from occurring.

By “topical administration” or “topical application” is meant non-systemic administration applied to body surfaces and includes the application of the compositions of the invention externally to the skin or mucosa as well as to different body cavities and where it does not significantly enter the blood stream.

“Alkyl” refers to a straight or branched hydrocarbon chain radical consisting of carbon and hydrogen atoms, containing no unsaturation, and which is attached to the rest of the molecule by a single bond. Alkyl groups preferably have from 1 to about 22 carbon atoms. One more preferred class of alkyl groups has from 1 to about 12 carbon atoms; and even more preferably from 1 to about 6 carbon atoms. Alkyl groups having 1, 2, 3, 4 or 5 carbon atoms are particularly preferred. Methyl, ethyl, n-propyl, iso-propyl and butyl, including n-butyl, tert-butyl, sec-butyl and iso-butyl are particularly preferred alkyl groups. As used herein, the term alkyl, unless otherwise stated, refers to both cyclic and noncyclic groups, although cyclic groups will comprise at least three carbon ring members, such as cyclopropyl or cyclohexyl. Alkyl radicals may be optionally substituted by one or more substituents, such as an aryl group, like in benzyl or phenethyl.

“Alkenyl” and “Alkynyl” refer to a straight or branched hydrocarbon chain radical consisting of carbon and hydrogen atoms, containing at least one unsaturation (one carbon-carbon double or triple bond respectively) and which is attached to the rest of the molecule by a single bond. Alkenyl and alkynyl groups preferably have from 2 to about 22 carbon atoms. One more preferred class of alkenyl and alkynyl groups has from 2 to about 12 carbon atoms; and even more preferably from 2 to about 6 carbon atoms. Alkenyl and alkynyl groups having 2, 3, 4 or 5 carbon atoms are particularly preferred. The terms alkenyl and alkynyl as used herein refer to both cyclic and noncyclic groups, although cyclic groups will comprise at least three carbon ring members. Alkenyl and alkenyl radicals may be optionally substituted by one or more substituents.

“Aryl” refers to a radical derived from an aromatic hydrocarbon by removal of a hydrogen atom from a ring carbon atom. Suitable aryl groups in the present invention include single and multiple ring compounds, including multiple ring compounds that contain separate and/or fused aryl groups. Typical aryl groups contain from 1 to 3 separated and/or fused rings and from 6 to about 22 carbon ring atoms. Preferably aryl groups contain from 6 to about 10 carbon ring atoms. Aryl radicals may be optionally substituted by one or more substituents. Specially preferred aryl groups include substituted or unsubstituted phenyl, substituted or unsubstituted naphthyl, substituted or unsubstituted biphenyl, substituted or unsubstituted phenanthryl and substituted or unsubstituted anthryl.

“Heterocyclyl” refers to a cyclic radical having as ring members atoms of at least two different elements. Suitable heterocyclyl radicals include heteroaromatic and heteroalicyclic groups containing from 1 to 3 separated and/or fused rings and from 5 to about 18 ring atoms. Preferably heteroaromatic and heteroalicyclic groups contain from 5 to about 10 ring atoms. Heterocycles are described in: Katritzky, Alan R., Rees, C. W., and Scriven, E. Comprehensive Heterocyclic Chemistry (1996) Pergamon Press; Paquette, Leo A.; Principles of Modern Heterocyclic Chemistry W. A. Benjamin, New York, (1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28. Suitable heteroaromatic groups in the compounds of the present invention contain one, two or three heteroatoms selected from N, O or S atoms and include, e.g., coumarinyl including 8-coumarinyl, quinolyl including 8-quinolyl, isoquinolyl, pyridyl, pyrazinyl, pyrazolyl, pyrimidinyl, furyl, pyrrolyl, thienyl, thiazolyl, isothiazolyl, triazolyl, tetrazolyl, isoxazolyl, oxazolyl, imidazolyl, indolyl, isoindolyl, indazolyl, indolizinyl, phthalazinyl, pteridinyl, purinyl, oxadiazolyl, thiadiazolyl, furazanyl, pyridazinyl, triazinyl, cinnolinyl, benzimidazolyl, benzofuranyl, benzofurazanyl, benzothienyl, benzothiazolyl, benzoxazolyl, quinazolinyl, quinoxalinyl, naphthyridinyl, and furopyridinyl. Suitable heteroalicyclic groups in the compounds of the present invention contain one, two or three heteroatoms selected from N, O or S atoms and include, e.g., pyrrolidinyl, tetrahydrofuryl, dihydrofuryl, tetrahydrothienyl, tetrahydrothiopyranyl, piperidyl, morpholinyl, thiomorpholinyl, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridyl, 2-pyrrolinyl, 3-pyrrolinyl, indolinyl, 2H-pyranyl, 4H-pyranyl, dioxanyl, 1,3 -dioxolanyl, pyrazolinyl, dithianyl, dithiolanyl, dihydropyranyl, dihydrothienyl, pyrazolidinyl, imidazolinyl, imidazolidinyl, 3-azabicyclo[3.1.0]hexyl, 3-azabicyclo[4.1.0]heptyl, 3H -indolyl, and quinolizinyl. Heterocylic radicals may be optionally substituted by one or more substituents.

The organic groups above defined may be substituted at one or more available positions by one or more suitable groups such as OR′, ═O, SR′, SOR′, SO₂R′, OSO₂R′, OSO₃R′, NO₂, NHR′, N(R′)₂, ═N—R′, N(R′)COR′, N(COR′)₂, N(R′)SO₂R′, N(R′)C(═NR′)N(R′)R′, CN, halogen, COR′, COOR′, OCOR′, OCOOR′, OCONHR′, OCON(R′)₂, CONHR′, CON(R′)₂, CON(R′)OR′, CON(R′)SO₂R′, PO(OR′)₂, PO(OR′)R′, PO(OR′)(N(R′)R′), substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclyl, wherein each of the R′ groups is independently selected from the group consisting of hydrogen, OH, NO₂, NH₂, SH, CN, halogen, COH, COalkyl, COOH, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list.

“Halogen” substituents in the present invention include F, Cl, Br, and I.

As used herein, the term “pharmaceutically acceptable” refers to those compounds, materials, compositions, and/or dosage forms which are, within the scope of sound medical judgment, suitable for contact with the tissues of human beings without excessive toxicity, irritation, allergic response, or other problem complications commensurate with a reasonable benefit/risk ratio. In some embodiments, the term “pharmaceutically acceptable” means approved by a regulatory agency or listed in the European or U.S. Pharmacopeia, or other generally recognized international pharmacopeia for use particularly in humans.

The term “pharmaceutically acceptable salts” refers to any salt which, upon administration to the patient is capable of providing (directly or indirectly) a compound as described herein. The preparation of salts can be carried out by methods known in the art.

For instance, pharmaceutically acceptable salts of compounds provided herein are synthesized from the parent compound, which contains a basic or acidic moiety, by conventional chemical methods. Generally, such salts are, for example, prepared by reacting the free acid or base forms of these compounds with a stochiometric amount of the appropriate base or acid in water or in an organic solvent or in a mixture of both. Generally, nonaqueous media like ether, ethyl acetate, ethanol, 2-propanol or acetonitrile are preferred. Examples of the acid addition salts include mineral acid addition salts such as, for example, hydrochloride, hydrobromide, hydroiodide, sulfate, nitrate, phosphate, and organic acid addition salts such as, for example, acetate, trifluoroacetate, maleate, fumarate, citrate, oxalate, succinate, tartrate, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts. Since hydroxytyrosol has three hydroxyl groups, alkali addition salts are particularly preferred such as Na⁺ and NX₄⁺ (wherein X is independently selected from H or a C1-C4 alkyl group).

The compounds of the invention may be in crystalline form either as free compounds or as solvates (e.g. hydrates, alcoholates, particularly methanolates) and it is intended that both forms are within the scope of the present invention. Methods of solvation are generally known within the art. The compounds of the invention may present different polymorphic forms, and it is intended that the invention encompasses all such forms.

The term “prodrug” is used in its broadest sense and encompasses those derivatives that are converted in vivo to the compounds of the invention as a result of spontaneous chemical reaction(s), enzyme catalyzed chemical reaction(s), and/or metabolic chemical reaction(s). Prodrugs can serve to enhance solubility, absorption and lipophilicity to optimize drug delivery, bioavailability and efficacy. Examples of prodrugs include, but are not limited to, derivatives and metabolites of the compounds of formula I that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Typical examples of prodrugs of the compounds of formula I have biologically labile protecting groups on a functional moiety of its structure. Thus, prodrugs include compounds that can be oxidized, reduced, aminated, deaminated, esterified, deesterified, alkylated, dealkylated, acylated, deacylated, phosphorylated, dephosphorylated, or other functional group change or conversion involving forming or breaking chemical bonds on the prodrug, by either enzymatic action or by general acid or base solvolysis. Preferably, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger “Medicinal Chemistry and Drug Discovery 6^{th} ed. (Donald J. Abraham ed., 2001, Wiley) and “Design and Applications of Prodrugs” (H. Bundgaard ed., 1985, Harwood Academic Publishers). More particularly, a large number of structurally-diverse prodrugs of hydroxytyrosol have been described (Fernandez-Bolaños, J G, López O, Fernández-Bolaños J, Rodríguez Gutiérrez G. Hydroxytyrosol and derivatives: isolation, synthesis, and biological properties. Cur. Org. Chem. 12: 442-463, 2008), the entire disclosure of which is incorporated herein by reference.

Any compound that is a prodrug of a compound of formula (I) is within the scope and spirit of the invention. Any compound referred to herein is intended to represent such specific compound as well as certain variations or forms. In particular, compounds referred to herein may have asymmetric centres and therefore exist in different enantiomeric or diastereomeric forms. Thus, any given compound referred to herein is intended to represent any one of a racemate, one or more enantiomeric forms, one or more diastereomeric forms, and mixtures thereof. Likewise, stereoisomerism or geometric isomerism about the double bond is also possible, therefore in some cases the molecule could exist as (E)-isomer or (Z)-isomer (trans and cis isomers). If the molecule contains several double bonds, each double bond will have its own stereoisomerism, that could be the same as, or different to, the stereoisomerism of the other double bonds of the molecule. Furthermore, compounds referred to herein may exist as atropisomers. All the stereoisomers including enantiomers, diastereoisomers, geometric isomers and atropisomers of the compounds referred to herein, and mixtures thereof, are considered within the scope of the present invention.

Unless otherwise stated, the compounds comprised in the topical pharmaceutical compositions of the invention are also meant to include isotopically-labelled forms i.e. compounds which differ only in the presence of one or more isotopically-enriched atoms. For example, compounds having the present structures except for the replacement of at least one hydrogen atom by a deuterium or tritium, or the replacement of at least one carbon by ¹³C- or ¹⁴C-enriched carbon, or the replacement of at least one nitrogen by ¹⁵N-enriched nitrogen are within the scope of this invention.

The term “carrier” refers to a diluent, adjuvant, excipient or vehicle with which the active ingredient is administered. Suitable pharmaceutical carriers are described in “Remington's Pharmaceutical Sciences” by E. W. Martin, 1995.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

In one preferred embodiment of the present invention, the topical pharmaceutical composition comprises a compound of formula (I) wherein R₁, R₂ and R₃ are independently selected from the group consisting of hydrogen, substituted or unsubstituted C₁-C₂₂ alkyl, substituted or unsubstituted C₂-C₂₂ alkenyl, substituted or unsubstituted C₂-C₂₂ alkynyl, C₆-C₂₂ substituted or unsubstituted aryl, and substituted or unsubstituted heterocyclyl having from 5 to 18 ring atoms, ORₐ, SRₐ, SORₐ, SO₂Rₐ, OSO₂Rₐ, OSO₃Rₐ, NO₂, NHRₐ, N(Rₐ)₂, ═N—Rₐ, N(Rₐ)CORₐ, N(CORₐ)₂, N(Rₐ)SO₂R′, N(Rₐ)C(═NRₐ)N(Rₐ)Rₐ, CN, halogen, CORₐ, COORₐ, OCORₐ, OCOORₐ, OCONHRₐ, OCON(Rₐ)₂, CONHRₐ, CON(Rₐ)₂, CON(Rₐ)ORₐ, CON(Rₐ)SO₂Rₐ, PO(ORₐ)₂, PO(ORₐ)Rₐ, PO(ORₐ)(N(Rₐ)Rₐ) and aminoacid ester;

And each of the Rₐ groups is independently selected from the group consisting of hydrogen, substituted or unsubstituted C₁-C₂₂ alkyl, substituted or unsubstituted C₂-C₂₂ alkenyl, substituted or unsubstituted C₂-C₂₂ alkynyl, substituted or unsubstituted C₆-C₂₂ aryl, and substituted or unsubstituted heterocyclyl having from 5 to 18 ring atoms.

Preferred compounds of formula (I) are hydroxytyrosol (R₁, R₂, R₃ are H) and the following hydroxytyrosol derivatives:

(1) carboxylic acid esters, such as acetate (R₁, R₂, are H and R₃ is —COCH₃),

(2) sulphonate esters, such as alkyl- or aralkylsulphonyl (for example, methanesulphonyl);

(3) phosphate esters;

(4) phosphonate esters;

(5) phosphoramidate esters;

(6) amino acid esters (for example, alanine, L-valyl or L-isoleucyl);

Thus, in one more preferred embodiment of the present invention, the compound is selected from a compound of formula (I) wherein R₁, R₂ and R₃ are independently selected from the group consisting of hydrogen, SO₂Rₐ, CORₐ, PO(ORₐ)₂, PO(ORₐ)Rₐ, PO(ORₐ)(N(Rₐ)Rₐ) and aminoacid ester;

and each of the Rₐ groups is independently selected from the group consisting of hydrogen, substituted or unsubstituted C₁-C₂₂ alkyl, substituted or unsubstituted C₂-C₂₂ alkenyl, substituted or unsubstituted C₂-C₂₂ alkynyl, substituted or unsubstituted C₆-C₂₂ aryl, and substituted or unsubstituted heterocyclic group having from 5 to 18 ring atoms.

Some specific most preferred compounds of formula (I) in the present invention are for example the following:
Hydroxytyrosol (R₁, R₂, R₃ are H)
Hydroxytyrosol acetate (R₁, R₂, are H and R₃ is —COCH₃).

In additional preferred embodiments, the preferences described above for the different substituents are combined.

As noted above, the topical pharmaceutical compositions of the present invention are useful as microbicides for preventing sexually transmitted diseases caused by fungi, bacteria or viruses, preferably HIV.

For example, the microbicide compositions of the present invention may be useful to people who are HIV-positive in several ways. Since microbicides neutralize disease causing organisms in both semen and vaginal secretions, they may give HIV-positive users a way of reducing their partner's risk of contracting HIV during sex. Likewise, a pharmaceutical composition according to the present invention could also reduce the risk of two HIV-positive partners being re-infected with different strains of HIV. They may also reduce an HIV-positive person's risk of getting other STDs, bladder infections, or yeast infections. For people with compromised immune systems, this could be an important advantage. And importantly, the pharmaceutical composition of the present invention can protect women herselves against HIV infection, her newborn children and further propagation of the HIV infection to other via intercourse.

Moreover, hydroxytyrosol and its derivatives are commercially available or easily accessible for example by known synthetic methods. Therefore, the microbicide compositions of these compounds are a cheap and easy way to use as prevention method against HIV-transmission and infection.

Administration of the topical pharmaceutical compositions of the present invention may be by any suitable method, such as oral, rectal, and vaginal administration. In a preferred embodiment, the pharmaceutical composition is topically applied to a body cavity such as the vagina, anus, or the mouth.

As noted hereinafter, the compositions of the present invention may be in the form of foams, creams, ointments, gels, jellies, suppositories, tablets, aerosols, gargles, mouthwashes, microemulsions, depot devices, etc. In one particular embodiment, gels, creams or foams are preferred. Most preferred are vaginal creams. In another embodiment the composition is in the form of a slow release formulation, preferably in the form of a device, such as for example a vaginal ring. In another particular embodiment, for example for rectal administration, suppositories are preferred. Likewise, as described hereinafter, the compositions of the present invention can be incorporated in different articles such as an intrauterine device (IUD), vaginal diaphragm, vaginal sponge, pessary, condom, etc. Among them, condoms are especially preferred.

Creams or lotions are oil-in-water emulsions. Oily bases that can be used are fatty alcohols, especially those containing from 12 to 18 carbon atoms, for example lauryl, cetyl or stearyl alcohol, fatty acids, especially those containing from 10 to 18 carbon atoms, for example palmitic or stearic acid, fatty acid esters, e.g. glyceryl tricaprilocaprate (neutral oil) or cetyl palmitate, liquid to solid waxes, for example isopropyl myristate, wool wax or beeswax, and/or hydrocarbons, especially liquid, semi-solid or solid substances or mixtures thereof, for example petroleum jelly (petrolatum, Vaseline) or paraffin oil. Suitable emulsifiers are surface-active substances having predominantly hydrophilic properties, such as corresponding non-ionic emulsifiers, for example fatty acid esters of polyalcohols and/or ethylene oxide adducts thereof; especially corresponding fatty acid esters with (poly)ethylene glycol, (poly)propylene glycol or sorbitol, the fatty acid moiety containing especially from 10 to 18 carbon atoms, especially partial glycerol fatty acid esters or partial fatty acid esters of polyhydroxyethylene sorbitan, such as polyglycerol fatty acid esters or polyoxyethylene sorbitan fatty acid esters (Tweens), and also polyoxyethylene fatty alcohol ethers or fatty acid esters, the fatty alcohol moiety containing especially from 12 to 18 carbon atoms and the fatty acid moiety especially from 10 to 18 carbon atoms, such as polyhydroxyethyleneglycerol fatty acid ester (for example Tagat S), or corresponding ionic emulsifiers, such as alkali metal salts of fatty alcohol sulfates, especially having from 12 to 18 carbon atoms in the fatty alcohol moiety, for example sodium lauryl sulfate, sodium cetyl sulfate or sodium stearyl sulfate, which are usually used in the presence of fatty alcohols, for example cetyl alcohol or stearyl alcohol. Additives to the aqueous phase are, inter alia agents that prevent the creams from drying out, for example humectants, such as polyalcohols, such as glycerol, sorbitol, propylene glycol and/or polyethylene glycols, and also preservatives, perfumes, gelling agents, etc.

Ointments are water-in-oil emulsions that contain up to 70%, but preferably from approximately 20% to approximately 50%, water or aqueous phase. Suitable as fatty phase are especially hydrocarbons, for example petroleum jelly, paraffin oil and/or hard paraffins, which, in order to improve the water-binding capacity, preferably contain suitable hydroxy compounds, such as fatty alcohols or esters thereof, for example cetyl alcohol or wool wax alcohols, or wool wax or beeswax. Emulsifiers are corresponding lipophilic substances, for example of the type indicated above, such as sorbitan fatty acid esters (Spans), for example sorbitan oleate and/or sorbitan isostearate. Additives to the aqueous phase are, inter alia humectants, such as polyalcohols, for example glycerol, propylene glycol, sorbitol and/or polyethylene glycol, and also preservatives, perfumes, etc.

Microemulsions are isotropic systems based on the following four components: water, a surfactant, for example a tensioactive, a lipid, such as a non-polar or polar oil, for example paraffin oil, natural oils such as olive or maize oil, and an alcohol or polyalcohol containing lipophilic groups, for example 2-octyldodecanol or ethoxalated glycerol or polyglycerol esters. If desired, other additives may be added to the microemulsions. Microemulsion have micelles or particles with sizes below 200 nm and are transparent or translucid systems, the form spontaneoulsy and are stable.

Fatty ointments are water-free and contain as base especially hydrocarbons, for example paraffin, petroleum jelly and/or liquid paraffins, also natural or partially synthetic fat, such as fatty acid esters of glycerol, for example coconut fatty acid triglyceride, or preferably hardened oils, for example hydrogenated groundnut oil, castor oil or waxes, also fatty acid partial esters of glycerol, for example glycerol mono- and di-stearate, and also, for example, the fatty alcohols increasing the water-absorption capacity, emulsifiers and/or additives mentioned in connection with the ointments.

With gels, a distinction is made between aqueous gels, water-free gels and gels having a low water content, which gels consist of swellable, gel-forming materials. There are used especially transparent hydrogels based on inorganic or organic macromolecules. High molecular weight inorganic components having gel-forming properties are predominantly water-containing silicates, such as aluminium silicates, for example bentonite, magnesium aluminium silicates, for example Veegum, or colloidal silicic acid, for example Aerosil. As high molecular weight organic substances there are used, for example, natural, semisynthetic or synthetic macromolecules. Natural and semi synthetic polymers are derived, for example, from polysaccharides containing a great variety of carbohydrate components, such as celluloses, starches, tragacanth, gum arabic and agar-agar, and gelatin, alginic acid and salts thereof, for example sodium alginate, and derivatives thereof, such as lower alkylcelluloses, for example methyl- or ethyl-cellulose, carboxy- or hydroxy-lower alkylcelluloses, for example carboxymethyl- or hydroxyethyl-cellulose. The components of synthetic gel-forming macromolecules are, for example, suitably substituted unsaturated aliphatic compounds such as vinyl alcohol, vinylpyrrolidine, acrylic or methacrylic acid.

Emulsion-gels—also called “emulgels”—represent topical compositions which combine the properties of a gel with those of an oil-in-water emulsion. In contrast to gels, they contain a lipid phase which due to its fat-restoring properties enables the formulation to be massaged in whilst, at the same time, the direct absorption into the skin is experienced as a pleasant property. Furthermore, one can observe an increased solubility for lipophilic active ingredients. One advantage of emulsion-gels over oil-in-water emulsions resides in the enhanced cooling effect which is brought about by the coldness due to evaporation of the additional alcohol component, if present.

Foams are administered, for example, from pressurised containers and are liquid oil-in water emulsions in aerosol form; unsubstituted hydrocarbons, such as alkanes, for example propane and/or butane, are used as propellant. As oil phase there are used, inter alia hydrocarbons, for example paraffin oil, fatty alcohols, for example cetyl alcohol, fatty acid esters, for example isopropyl myristate, and/or other waxes. As emulsifiers there are used, inter alia, mixtures of emulsifiers having predominantly hydrophilic properties, such as polyoxyethylene sorbitan fatty acid esters (Tweens), and emulsifiers having predominantly lipophilic properties, such as sorbitan fatty acid esters (Spans). The customary additives, such as preservatives, etc., are also added.

Tinctures and solutions generally have an ethanolic base, to which water may be added and to which there are added, inter alia, polyalcohols, for example glycerol, glycols and/or polyethylene glycol, as humectants for reducing evaporation, and fat-restoring substances, such as fatty acid esters with low molecular weight polyethylene glycols, propylene glycol or glycerol, that is to say lipophilic substances that are soluble in the aqueous mixture, as a replacement for the fatty substances removed from the skin by the ethanol, and, if necessary, other adjuncts and additives. Suitable tinctures or solutions may also be applied in spray form by means of suitable devices.

The compositions according to the invention may also comprise conventional additives and adjuvants for topical applications, such as preservatives, especially paraben esters like methylparaben, ethylparaben, propylparaben, butylparaben, or quaternary ammonium compounds like benzalkonium chloride, or formaldehyde donors like imidazonidinyl urea, or alcohols like benzyl alcohol, phenoxyethanol or acids like benzoic acid, sorbic acid; acids or bases used as pH buffer excipients; antioxidants, especially phenolic antioxidants like hydroquinone, tocopherol and derivatives thereof, as well as flavonoids, or miscellaneous antioxidants like ascorbic acid,ascorbyl palmitat; perfumes; fillers such as kaolin or starch; pigments or colorants; UV-screening agents; moisturizers, especially glycerin, butylen glycol, hexylen glycol, urea, hyaluronic acid or derivatives thereof; anti-free radical agents such as vitamin E or derivatives thereof; penetration enhancers especially propylene glycol; ethanol; isopropanol; dimethylsulfoxide; N-methyl-2-pyrrolidone; fatty acids/alcohols such as oleic acid, oleyl alcohol; terpenes such as limonen, menthol, 1-8 cineole; alkyl esters such as ethyl acetate, butyl acetate; ion pairing agents such as salicylic acid.

Further details concerning suitable topical formulations may be obtained by reference to standard textbooks such as Banker and Rhodes (Ed) Modern Pharmaceutics 4^{th} ed. (2002) published by Marcel Dekker Inc.; Harry's Cosmeticology (2000), 8th Edition, Chemical Publishing Co.; Remington's Pharmaceutical Sciences 20^{th} ed Mack Publishing Co.(2000).

Vaginal Administration

The pharmaceutical composition of the invention may be applied to the vagina in a number of forms including aerosols, foams, sprays, pastes, gels, jellies, creams, suppositories, tablets, pessaries, tampons, devices such as vaginal rings, etc. They can be in the form of immediate release or controlled release. Foams, creams and gels are preferred forms. Compositions suitable for application to the vagina are disclosed in U.S. Pat. Nos. 2,149,240, 2,330,846, 2,436,184, 2,467,884, 2,541,103, 2,623,839, 2,623,841, 3,062,715, 3,067,743, 3,108,043, 3,174,900, 3,244,589, 4,093,730, 4,187,286, 4,283,325, 4,321,277, 4,368,186, 4,371,518, 4,389,330, 4,415,585, and 4,551,148, which are incorporated herein by reference, and the present method may be carried out by applying the pharmaceutical composition to the vagina in the form of such a composition.

In a particularly preferred embodiment, the pharmaceutical composition is topically applied to the vagina. Thus, the present method may involve topical application to the vagina to prevent HIV infection or other STDs as a result of vaginal intercourse. Typically, the topical application is carried out prior to the beginning of vaginal intercourse, suitably 0 to 60 minutes, preferably 0 to 5 minutes, prior to the beginning of vaginal intercourse. The application may be carried out into and around the vagina and vaginal area (e.g., the individual anatomical parts, such as, labia majora, labia minora, clitoris, etc.) of a female.

The composition of the invention containing the compound of formula (I) may be applied to the vagina in any conventional manner. Suitable devices for applying the composition to the vagina are disclosed in U.S. Pat. Nos. 3,826,828, 4,108,309, 4,360,013, and 4,589,880, which are incorporated herein by reference.

Pharmaceutical creams, as known in the art, are viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant.

As defined herein, a “vaginal cream” is a semi-solid preparation suitable for application to the vaginal tract. Various classes of excipients or vehicles can be used in the vaginal cream and are known to those in the art. The excipients comprise materials of naturally occurring or synthetic origin that do not adversely affect the components of the formulation. Suitable carriers for use herein include but are not limited to purified water, white soft paraffin, mucoadhesive polymers, liquid paraffin, polysorbate 60, sorbitan stearate silicone, waxes, petroleum, jelly, polyethylene glycol, and a variety of other materials, depending on the specific type of formulation used.

A preferred class of bioadhesive gelling polymers to be used in this invention is that comprised of acrylic acid polymers crosslinked with allyl sucrose or allyl ethers of pentaerythritol, commercially available with the name Carbopol (Carbomer) from B.F. Goodrich Chemical Co. Carbopol 934P and 971P are usually considered the ideal candidate for vaginal administration.

Another preferred class of bioadhesive gelling polymer to be used in the present invention is that comprised of acrylic acid polymers crosslinked with divinyl glycol, commercially available with the trademark Noveon AA-1 Polycarbophil USP (Polycarbophil AA1).

For example, suitable vehicle bases include, but are not limited to, hydrocarbon bases or oleaginous bases, absorption bases, water-removable bases and water-soluble bases. In some embodiments, the vehicle base is non-irritating, non-staining, stable, non-pH dependent and/or compatible with the compound of formula (I).

Rectal Administration

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of the active ingredient with the softened or melted carrier(s) followed by chilling and shaping in moulds.

In another embodiment, the present invention involves topical administration of the composition to the anus. The composition administered to the anus is suitably a foam, cream, jelly, etc., such as those described above with regard to vaginal application. In the case of anal application, it may be preferred to use an applicator which distributes the composition substantially evenly throughout the anus. For example, a suitable applicator is a tube 2.5 to 25 cm, preferably 5 to 10 cm, in length having holes distributed regularly along its length.

Oral Administration

In another embodiment, the present method may be carried out by applying the pharmaceutical composition orally. Oral application is suitably carried out by applying a composition which is in the form of a mouthwash or gargle. Oral application is especially preferred to prevent infection during dental procedures. Suitably, the composition is applied just prior to the beginning of the dental procedure and periodically throughout the procedure. Formulations suitable for topical administration in the mouth include lozenges comprising the active ingredient in a flavored base, usually sucrose and acacia or tragacanth; pastilles comprising the active ingredient in an inert basis such as gelatin and glycerin, or sucrose and acacia; and mouthwashes comprising the active ingredient in a suitable liquid carrier.

In the case of a mouthwash or gargle, it may be preferred to include in the composition an agent which will mask the taste and/or odor of the compound of formula (I). Such agents include those flavoring agents typically found in mouthwashes and gargles, such as spearmint oil, cinnamon oil, etc.

Concentrations, Release and Articles

The therapeutically effective amount of the compound or compounds of formula (I) to be administered will generally depend, among other factors, on the chosen method of administration. For this reason, the doses mentioned in this invention must only be considered as guidelines for the person skilled in the art. It is understood that animal studies may be performed to determine an appropriate dosage amount.

It is noted that when the composition is in the form of a suppository (including vaginal suppositories), the suppository will usually be 1 to 5 grams, preferably about 3 grams, and the entire suppository will be applied. A vaginal tablet will suitably be 1 to 5 grams, preferably about 2 grams, and the entire tablet will be applied. When the composition is vaginal cream, suitably 0.1 to 2 grams, preferably about 0.5 grams of the cream will be applied. When the composition is a water-soluble vaginal cream, suitably 0.1 to 2 grams, preferably about 0.6 grams, are applied. When the composition is a vaginal spray-foam, suitably 0.1 to 2 grams, preferably about 0.5 grams, of the spray-foam are applied. When the composition is an anal cream, suitably 0.1 to 2 grams, preferably about 0.5 grams of the cream is applied. When the composition is an anal spray-foam, suitably 0.1 to 2 grams, preferably about 0.5 grams of the spray-foam are applied. When the composition is a mouthwash or gargle, suitably 1 to 10 ml, preferably about 5 ml are applied.

Further, the amount of the compound or compounds of formula (I) in a dosage form must be such to achieve an effective local anal, oral or vaginal concentration, that is to say, the compound(s) of formula (I) must be present at a level sufficient to originate a microbicide effect upon administration.

In a preferred embodiment of the invention, the pharmaceutical composition comprises from about 0.1 μg to about 300 mg of a compound of formula (I), or a pharmaceutically acceptable salt, solvate, prodrug or isomer thereof. More preferred, the composition comprises from about 1 μg to about 30 mg of a compound of formula (I), or a pharmaceutically acceptable salt, solvate, prodrug or isomer thereof.

The present compositions may also be in the form of a time-release composition. In this embodiment, the compound of formula (I) is incorporated in a composition which will release the active ingredient at a rate which will result in an effective vaginal or anal concentration of said compound of formula (I). Time-release compositions are disclosed in Controlled Release of Pesticides and Pharmaceuticals, D. H. Lew, Ed., Plenum Press, New York, 1981; and U.S. Pat. Nos. 5,185,155; 5,248,700; 4,011,312; 3,887,699; 5,143,731; 3,640,741; 4,895,724; 4,795,642; Bodmeier et al, Journal of Pharmaceutical Sciences, vol. 78 (1989); Amies, Journal of Pathology and Bacteriology, vol. 77 (1959); and Pfister et al, Journal of Controlled Release, vol. 3, pp. 229-233 (1986), all of which are incorporated herein by reference.

The present compositions may also be in the form which releases the compound of formula (I) in response to some event such as vaginal or anal intercourse. For example, the composition may contain the compound of formula (I) in vesicles or liposomes, which are disrupted by the mechanical action of intercourse. Compositions comprising liposomes are described in U.S. Pat. No. 5,231,112 and Deamer and Uster, “Liposome Preparation: Methods and Mechanisms”, in Liposomes, pp. 27-51 (1983); Sessa et al, J. Biol. Chem., vol. 245, pp. 3295-3300 (1970); Journal of Pharmaceutics and Pharmacology, vol. 34, pp. 473-474 (1982); and Topics in Pharmaceutical Sciences, D. D. Breimer and P. Speiser, Eds., Elsevier, New York, pp. 345-358 (1985), which are incorporated herein by reference.

It should also be realized that the present compositions may be associated with an article, such as an intrauterine device (IUD), vaginal diaphragm, vaginal ring, vaginal sponge, pessary, condom, etc. In the case of an IUD or diaphragm, time-release and/or mechanical-release compositions may be preferred, while in the case of condoms, mechanical-release compositions are preferred.

In another embodiment, the present invention provides novel articles, which are useful for the prevention of HIV infection. In particular, the present articles are those which release the compound of formula (I) when placed on an appropriate body part or in an appropriate body cavity. Thus, the present invention provides IUDs, vaginal diaphragms, vaginal sponges, pessaries, or condoms which contain or are associated with a compound of formula (I).

Thus, the present article may be an IUD which contains one or more compounds of formula (I). Suitable IUDs are disclosed in U.S. Pat. Nos. 3,888,975 and 4,283,325 which are incorporated herein by reference. The present article may be an intravaginal sponge which comprises and releases, in a time-controlled fashion, the compound of formula (I). Intravaginal sponges are disclosed in U.S. Pat. Nos. 3,916,898 and 4,360,013, which are incorporated herein by reference. The present article may also be a vaginal dispenser, which releases the compound of formula (I). Vaginal dispensers are disclosed in U.S. Pat. No. 4,961,931, which is incorporated herein by reference.

The present article may also be a condom which is coated with a compound of formula (I). In a preferred embodiment, the condom is coated with a lubricant or penetration enhancing agent which comprises a compound of formula (I) and an spermicide, which is optionally selected from benzalkonium chloride, benzethonium chloride, cetyl pyridinium chloride, methylbenzethonium chloride, tetra-decyltrimethyl ammonium bromide, benzalkonium bromide, monylphenyl ethers, lauryl ethers, and octoxynols. However, it is recommended that use of a condom should be associated with use of an appropriate lubricating agent, i.e. one that does not degrade the mechanical strength properties of the condom and that does not increase its porosity due to the latex being attacked. For example, EP-A-0 457 127 describes a lubricant based on silicone oil for treating the latex of condoms, EP-A-0 475 664 describes a lubricating composition and use thereof with condoms, and FR-A-2 666 587 describes a lubricant comprising polydimethylsiloxane. Other lubricants and penetration enhancing agents are described in U.S. Pat. Nos. 4,537,776; 4,552,872; 4,557,934; 4,130,667, 3,989,816; 4,017,641; 4,954,487; 5,208,031; and 4,499,154, which are incorporated herein by reference.

In another embodiment the compound of formula (I), more preferably hydroxytyrosol, is used in combination with other active ingredients, preferably microbiocides or anti-HIV agents. Remarkably, Hydroxytyrosol has shown lack of cross resistance against other anti HIV agents, and therefore is suitable for use in combination with other microbicides or antiviral agents.

The following examples further describe and demonstrate particular embodiments within the scope of the present invention. The examples are given solely for illustration and are not to be construed as limitations.

### EXAMPLES

Different formulations of 3,4-dihydroxyphenyl ethanol were successfully prepared.

### Example 1

### Vaginal Cream

One gram of vaginal cream is prepared having the following composition: 3,4-dihydroxyphenyl ethanol 50 mg, Sorbitan monostearate 45.0 mg, polysorbate 60 (Tween 60) 15.0 mg, Cetyl palmitate (Cutina CP-A) 30.0 mg, Viscous paraffin 130.46 mg, Cetylstearyl alcohol 100.0 mg, Hyaluronic acid 50.0 mg, Purified water 630.0 mg.

### Example 2

### Tablet Controlled Release Formulation

A tablet formulation for controlled release is prepared by wet granulation of the ingredients with purified water, followed by the addition of magnesium stearate and compression. The hypromellose can utilize varying viscosity grades.

One tablet of this formulation has the following composition: 3,4-dihydroxyphenyl ethanol 50 mg, hypromellose 112 mg, lactose monohydrate 53 mg, pregelatinized starch 28 mg, magnesium stearate 7 mg, purified water q.s.

Drug release takes place over a period of about 6-8 hours and is completed after 12 hours.

### Example 3

### Capsule Controlled Release Formulation

A capsule formulation for controlled release is prepared by wet granulation of ingredients a, b, c and e, and then extruding the material using an extruder, followed by spheronization of the extrudate and drying. The dried pellets are then coated with a release-controlling membrane (d) or polymer. The final product is filled into a two-piece, hard gelatin or hydroxypropyl methylcellulose capsule.

One capsule of this formulation has the following composition: (a) 3,4-dihydroxyphenyl ethanol 50 mg, (b) microcrystalline mellulose 125 mg, (c) lactose monohydrate 125 mg, (d) ethyl cellulose 13 mg, (e) purified water q.s., (f) gelatin capsules

### Example 4

### Oral Suspension

An oral suspension is prepared by admixing the active ingredient with the excipients and filled as dry powder. Purified water is added and mixed well before use.

The oral suspension has the following composition: 3,4-dihydroxyphenyl ethanol 50 mg, confectioner's sugar 2000 mg, simethicone 300 mg, methylparaben 30 mg, propylparaben 10 mg, flavor 500 mg, purified water q.s. to 5.00 ml.

### Example 5

### Suppository

A suppository is prepared by the following procedure: one-fifth of the Witepsol H15 is melted in a steam-jacketed pan at 45° C. maximum. The active ingredient is sifted through a 200 micron sieve and added to the molten base with mixing, using a Silverson fitted with a cutting head, until a smooth dispersion is achieved. Maintaining the mixture at 45° C., the remaining Witepsol H15 is added to the suspension and stirred to ensure a homogenous mix. The entire suspension is passed through a 250 micron stainless steel screen and, with continuous stirring, is allowed to cool to 40° C. At a temperature of 38° C. to 40° C., 2.02 g of the mixture is filled into suitable, 2 ml plastic molds. The suppositories are allowed to cool to room temperature.

The suppository contain: 3,4-dihydroxyphenyl ethanol 50 mg, Hard Fat B.P. (Witepsol H15-Dynamit Nobel) 1770.

### Example 6

### Vaginal Suppository

A vaginal suppository is prepared having the following composition: 3,4-dihydroxyphenyl ethanol 50 mg, Hexanetriol 100 mg, polyethylene glycol 1500 q.s.

### Example 7

### Bioadhesive Vaginal Cream

A bioadhesive vaginal cream with spermicide is prepared having the following composition: 3,4-dihydroxyphenyl ethanol 50 mg, Nonoxynol 9 80 mg, Carbopol 971P mg %, Polycarbophil AA-1 15 mg, glycerine 100 mg, Cremophor 100 mg, NaOH qs pH 4.5, purified water up to 1 g

### Example 8

### Vaginal Spray-Foam

A vaginal spray-foam is prepared having the following composition: 3,4-dihydroxyphenyl ethanol 50 mg, polyethylene glycol 6000 2 g, nonionic emulsifying agent 2 g, Water 85 g, Freon 12/144 (70:30) 10 g

### Example 9

### Vaginal Gel

A vaginal gel is prepared by the following procedure: Sodium hydroxide and hydrochloric acid are used as 10% w/w solutions to adjust pH to a target of 4.4. The methylparaben and propylparaben are dissolved in heated glycerin. Hydroxyethylcellulose is added and dispersed to form an organic phase. Edetate disodium and citric acid are dissolved in purified water, tenofovir is added and dispersed, pH adjusted to 4.5, and solution clarified by passage through a 0.22 μm filter. Aqueous and organic phases are mixed, stirred well then filled into tubes or applicators.

The vaginal gel has the following composition: 3,4-dihydroxyphenyl ethanol 50 mg, hydroxyethylcellulose NF (Natrasol (R) 250H) 25 mg, propylparaben NF 0.2 mg, methylparaben, NF 1.8 mg, edetate disodium, USP 0.5 mg, glycerin USP 200 mg, Citric Acid USP 10 mg, purified water, USP qs to 1 g.

### Example 10

### HT (Hydroxytyrosol) Evaluation on HIV-1 Replication

Antiviral Activity in Cell Line MT-2

The limphoblastoid cell line MT-2 was used as infection target, and recombinant virus NL4.3-Renilla as X4-tropic virus with the indicator gene Renilla, and recombinant virus NL4.3-delta-env-VSV-Luc as HIV VSV pseudotyped virus. This last vector generates virus able to enter the host cell independently of receptors.

The assays were performed pretreating the cell culture (100.000 cells/well in 96 well plates) at different concentrations of hydroxytyrosol. After 1 hour, the cell culture is infected with the recombinant viruses. For the viability assay, RPMI is used instead of viruses. The cell culture is maintained at 37° C. and 5% CO₂. After 48 hour the cells are lysated and the Renilla or luciferase activity, or cell viability respectively is measured.

The results are shown in FIG. 1. Further experiments were carried out after correcting the concentrations in infections with virus X4 tropic (NL4.3-ren) and VSV (Delta Luc), they are shown in FIG. 2.

It can be seen that Hydroxytyrosol has antiviral activity in the micromolar range, around 50-100 μM. The antiviral activity is specific and not due to toxicity. There seems to be a difference between viruses with different tropisms. The viral entry into the host cell does not appear to be the target of action of hydrxytyrosol, since it inhibits VSV pseudotyped viruses.

Antiviral Activity in Limphocytes of Peripheral Blood

Similar assays were carried out using peripheral blood mononuclear cells (PBMC's) for healthy donors, prior activation with PHA+IL-2 during 48 hours and maintaining with IL-2 for at least another 48 hours. In this case the number of cells per well was 250.000. In addition, infection with HIV virus with R5-tropism (JR-Ren) was also included in the assays.

The results are shown in FIG. 3. They confirm the activity of hydroxytyrosol in PBCM's, although the IC50 value for infection with X4-tropic is higher than in the assays with cell line MT-2, whereas the values in R5-tropic viruses (JR-Ren) and pseudotyped with VSV (Delta Luc) are similar to thos obtained in the cell line MT-2.

### Example 11

### HT (Hydroxytyrosol) Antiviral Activity in Trans-Infection Mediated by DC-SIGN

One of the most important mechanisms of HIV infection is that mediated by the introduction of the virus through cells presenting antigen DC-SIGN+, which is called trans-infection. This mechanism was studied with a system based on cells DC-SIGN+ (RAJI DC-SIGN) that mimetizes the natural infection conditions. The target of the infection are PBMC cells from healthy donors. The results obtained with Hydroxytyrosol are show in table 1 and FIG. 4.

TABLE 1

| | JR-Renilla |
|---|---|
| Sigmoidal dose-response | |
| Best-fit values | |
| BOTTOM | 21.1 |
| TOP | 103 |
| LOGEC50 | 0.6888 |
| EC50 | 4.884 |
| Std. Error | |
| LOGEC50 | 0.09743 |
| 95% Confidence Intervals | |
| EC50 | 2.821 to 8.456 |
| Goodness of Fit | |
| R² | 0.9867 |

As shown in FIG. 4, the trans-infection with R5-tropic virus is inhibited by hydroxytyrosol, with more potency that in the assays with direct infection.

### Example 12

### HT (Hydroxytyrosol) Antiviral Activity Against Raltegravir Resistant Viruses

The mechanism of action of hydroxytyrosol is not related with interference with viral entry, but it takes place in the initial steps of infection. There appears to be an inhibition by hydroxytyrosol of the HIV integration process.

Raltegravir is a commercial integase inhibitor, and eviltegravir is under development.

To assess the cross resistance of hydroxytyrosol we generated viruses resistant to raltegravir and eviltegravir through the introduction of mutation 148 (via directed mutagenesis) and we evaluated the susceptibility to hydroxytyrosol and raltegravir.

FIG. 5 shows the antiviral activity of hydroxytyrosol against wild type and raltegravir resistant HIV virus.

FIG. 6 shows the antiviral activity of raltegravir against wild type and raltegravir resistant HIV virus.

Surprisingly, hydroxytyrosol mantained its activity against integrase resistant viruses, unlike raltegravir that was used as control.

Therefore, hydroxytyrosol would be useful in combination with other agents, or as an additional drug for patients having developed integrase resistance.

### Example 13

### Safety of Hydroxytyrosol After Repeated Topical Administration

To determine the safety of hydroxytyrosol, a study of vaginal tolerance in rabbits after repeated once daily administration during 7 days was performed. Hydroxytyrosol was applied as a solution.

Under the conditions of the experiment, the topical administration of the HTS solution at two different concentrations of 90 and 397 mg/L caused no morphological alterations in the vagina. The mean irritation value (MIV) obtained was 0.00 indicating no irritative response in the vaginal mucosa of the rabbit.

All publications and patent applications cited herein are incorporated by reference to the same extent as if each individual publication or patent application was specifically and individually indicated to be incorporated by reference.

Although certain embodiments have been described in detail above, those having ordinary skill in the art will clearly understand that many modifications are possible in the embodiments without departing from the teachings thereof. All such modifications are intended to be encompassed within the claims of the invention.

1. A topical pharmaceutical composition which comprises a compound of formula (I): 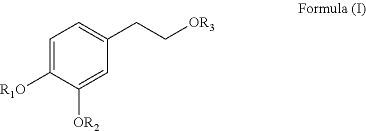
wherein
R₁, R₂ and R₃ are independently selected from the group consisting of hydrogen, substituted or unsubstituted alkyl, substituted or unsubstituted alkenyl, substituted or unsubstituted alkynyl, substituted or unsubstituted aryl, substituted or unsubstituted heterocyclyl, ORₐ, SRₐ, SORₐ, SO₂Rₐ, OSO₂Rₐ, OSO₃Rₐ, NO₂, NHR, N(Rₐ)₂, ═N—R, N(Rₐ)CORₐ, N(CORₐ)₂, N(Rₐ)SO₂R′, N(Rₐ)C(═NRₐ)N(Rₐ)Rₐ, CN, halogen, CORₐ, COORₐ, OCORₐ, OCOORₐ, OCONHRₐ, OCON(Rₐ)₂, CONHRₐ, CON(Rₐ)₂, CON(Rₐ)ORₐ, CON(Rₐ)SO₂Rₐ, PO(ORₐ)₂, PO(ORₐ)Rₐ, PO(ORₐ)(N(Rₐ)Rₐ) and aminoacid ester;
each of the Rₐ groups is independently selected from the group consisting of hydrogen, substituted or unsubstituted alkyl, substituted or unsubstituted alkenyl, substituted or unsubstituted alkynyl, substituted or unsubstituted aryl, and substituted or unsubstituted heterocyclyl,
or a pharmaceutically acceptable salt, solvate, prodrug or isomer thereof,
and a pharmaceutically acceptable carrier.

2. The topical pharmaceutical composition according to claim 1 wherein:
R₁, R₂ and R₃ are independently selected from the group consisting of hydrogen, substituted or unsubstituted C₂-C₂₂ alkyl, substituted or unsubstituted C₂-C₂₂ alkenyl, substituted or unsubstituted C₂-C₂₂ alkynyl, substituted or unsubstituted C₆-C₂₂ aryl, substituted or unsubstituted heterocyclyl having from 5 to 18 ring atoms, ORₐ, SRₐ, SORₐ, SO₂Rₐ, OSO₂Rₐ, OSO₃Rₐ, NO₂, NHRₐ, N(Rₐ)₂, ═N—Rₐ, N(Rₐ)CORₐ, N(CORₐ)₂, N(Rₐ)SO₂R′, N(Rₐ)C(═NR,)N(Rₐ)Rₐ, CN, halogen, CORₐ, COORₐ, OCORₐ, OCOORₐ, OCONHRₐ, OCON(Rₐ)₂, CONHRₐ, CON (Rₐ)₂, CON(Rₐ)ORₐ, CON(Rₐ)SO₂Rₐ, PO(ORₐ)₂, PO(ORₐ)Rₐ, PO(ORₐ)(N(Rₐ)Rₐ) and aminoacid ester;
and each of the Rₐ groups is independently selected from the group consisting of hydrogen, substituted or unsubstituted C₁-C₂₂ alkyl, substituted or unsubstituted C₂-C₂₂ alkenyl, substituted or unsubstituted C₂-C₂₂ alkynyl, substituted or unsubstituted C₆-C₂₂ aryl, and substituted or unsubstituted heterocyclyl having from 5 to 18 ring atoms.

3. The topical pharmaceutical composition according to claim 2, wherein R₁, R₂ and R₃ are independently selected from the group consisting of hydrogen, SO₂Rₐ, CORₐ, PO(ORₐ)₂, PO(ORₐ)Rₐ, PO(ORₐ)(N(Rₐ)Rₐ) and aminoacid ester.

4. The topical pharmaceutical composition according to claim 3, wherein the compound of formula (I) is hydroxytyrosol or hydroxytyrosol acetate.

5. The topical pharmaceutical composition according to claim 1 which comprises from 0.1 μg to 300 mg of a compound of formula (I).

6. The topical pharmaceutical composition according to claim 5 which comprises from 1 μg to 30 mg of a compound of formula (I).

7. The topical pharmaceutical composition according to claim 1 wherein said composition is a cream, a gel, a foam, a device or a suppository.

8. The topical pharmaceutical composition according to claim 1 formulated for use in topical vaginal application.

9. The topical pharmaceutical composition according to claim 8 wherein said composition is a vaginal cream or a vaginal device

10. A condom coated with a pharmaceutical composition as defined in claim 1.

11. (canceled)

12. A method for the prevention of sexually transmitted diseases (STDs), preferably HIV-infection comprising topically applying the topical pharmaceutical composition of claim 1 in an amount effective to prevent the STD.

13. (canceled)

14. The method of claim 12, wherein the topical pharmaceutical composition further comprises a microbicide or an antiretroviral agent.

15. (canceled)

16. The topical pharmaceutical composition of claim 1, further comprising a microbicide or an antiretroviral agent.

17. The topical pharmaceutical composition of claim 4, further comprising a microbicide or an antiretroviral agent.

18. The method of claim 12, wherein the STD is HIV infection.

19. The method of claim 12, wherein the topical pharmaceutical compostion comprises hydroxytyrosol or hydrosytyrosol acetate.

20. The method of claim 14, wherein the topical pharmaceutical compostion comprises hydroxytyrosol or hydrosytyrosol acetate.

21. The method of claim 18, wherein the topical pharmaceutical compostion comprises hydroxytyrosol or hydrosytyrosol acetate.
